(19) United States
(12) Patent Application Publication
(10) Pub. No.: US 2012/0221041 A1
(43) Pub. Date: Aug. 30, 2012

(54) ARTERY COMPRESSOR

(75) Inventors: Erik HANSSON, Uppsala (SE); Erik Törnkvist, Uppsala (SE); Martin Tilly, Uppsala (SE)

(73) Assignee: ST. JUDE MEDICAL SYSTEMS AB

(21) Appl. No.: 13/035,146

(22) Filed: Feb. 25, 2011

(51) Int. Cl. A61B 17/132

(52) U.S. Cl. 606/203

(57) ABSTRACT

The present invention relates to a compression arrangement (1) adapted to be arranged around a patient's forearm (9) to provide pressure to an artery puncture site (10). The compression arrangement (1) comprises, a ventral part (3), adapted to be arranged at the ventral side (12) of the forearm (9), a dorsal part (4), adapted to be arranged at the dorsal side (18) of the forearm (9), an attachment band (2), and a compression element (5) in the shape of an inflatable bladder, adapted to provide pressure to the artery puncture site (10) when the compression element (5) is inflated. The ventral part (3) comprises a compression element part (6) where the compression element (5) is arranged, and the ventral part (3) is essentially rigid and has a predetermined shape adapted to the anatomy of the forearm (9). The dorsal part (4) is at least partly adapted to be supported against the forearm (9) by a supporting part (8), the ventral part (3) and the dorsal part (4) being interconnected by a flexible part (7), such that the ventral part (3), the flexible part (7) and the dorsal part (4) generally form a U-shape being elastic and form stable, and wherein the attachment band (2) is adapted to adjust the ventral and dorsal parts (3, 4) in relation to each other.

### FIELD OF THE INVENTION

The present invention relates to an artery compression arrangement, and also a method related to the arrangement, according to the preambles of the independent claims, with which artery compression arrangement a compression force is applied on the artery such that haemostasis can be obtained.

### BACKGROUND OF THE INVENTION

To access a patient's vascular system for an invasive medical procedure such as catheterization or similar procedures, a puncture is made in e.g. the femoral artery or the radial artery. The present invention is related to radial artery procedures. Following an invasive medical procedure, such as catheterisation or similar invasive medical procedure, the flow of blood through the puncture wound has to be stopped, so that haemostasis can begin as soon and fast as possible after the completion of the invasive medical procedure. Several devices have been suggested that facilitate and accelerate this haemostasis by providing a compression pressure that compresses blood vessels in various parts of the body to stop the flow of blood therethrough.

In the case of radial artery catheterisation, several radial artery occluders have been developed adapted to stop the flow of blood through the puncture wound in the wrist by applying a compression force that occludes the radial artery. An illustrative example of such a pressure-applying device is disclosed in U.S. Pat. No. 7,498,477, which discloses a haemostatic device with a flexible band adapted to be wrapped around a patient's limb where bleeding is to be stopped at a puncture site. The band has a curved plate that is transparent to ensure the puncture site being visible through the band. A main balloon is pressed against a puncture site of a patient and filled with a first fluid to inflate the balloon. A pressing member is further provided on the main balloon, and when filled with a second fluid, it presses against the main balloon to apply a compressive force to the puncture site that acts in an inclined direction with respect to the surface of the limb of the patient. The main balloon and the pressing member are held in place against the puncture site with a flexible band and a curved plate.

Another example of such a pressure-applying device is known from U.S. Pat. No. 5,601,597. This known artery occluder comprises a wrist splint, an adjustable securing strap attached to one end of the splint, and an adjustable pressure strap attached to the other end of the splint and provided with a pressure pad. When the artery occluder is mounted around the forearm of a patient, the wrist splint extends along the distal end of the forearm and the back of the wrist and hand, the securing strap extends around the palm of the hand, and the pressure strap extends around the distal end of the forearm, with the pressure pad being positioned over the puncture wound in the radial artery. During use of this occluder, the adjustable pressure strap is slowly tightened over the bleeding wound in the radial artery until the flow of blood in the radial artery has stopped at the wound. This aids haemostasis in the wound, but allows the ulnar artery to deliver enough blood to ensure tissue viability. In addition, the adjustable securing strap is tightened around the palm of the hand to help immobilize the wrist.

U.S. Pat. No. 6,647,986 shows a hand/wrist positioning splint to keep the hand positioned for radial artery access and to permit application of a haemostasis band. Two straps are used to secure the patient's hand to the splint and the haemostasis band comprises buckles to apply sufficient pressure to the puncture site.

From WO 96/25110 a further device for compression of an artery is known. The device comprises an elongated compression element, a pressure distribution and support plate and strap means for holding the compressing element and the pressure distribution plate in place.

Another compression device for radial artery is shown in NL 1016025. The device uses two bands to secure the wrist and a third band is used to stop bleeding at a puncture site.

And finally, in U.S. Pat. No. 4,798,199, an arterial wrist support is disclosed used to support a patient's extremity for arterial or intravenous care that includes a substantial rigid, unitary moulded body adapted to matingly engage the patient's hand, wrist and at least a portion of the patient's forearm.

The inventors of the present invention have identified a need for an improved compressor device which is easy to use and which provides for a more hygienic process than prior art devices. There is further a need for a compressor device which is economical to manufacture and ship to the end user.

It is an object of the invention to provide an improved compressor device, which facilitates and improves the procedure of artery compression and eliminates venous stasis, and which enables an accurately applied pressurization against a puncture site.

It is a further object of the invention to provide a compression arrangement, which enables a following adjustment of the applied pressure to the puncture site.

### SUMMARY OF THE INVENTION

The above-mentioned object is achieved by the present invention according to the independent claims.

Preferred embodiments are set forth in the dependent claims.

The compression arrangement adapted to be arranged around a patient's forearm to provide pressure to an artery puncture site, in accordance with the present invention, comprises a ventral part, adapted to be arranged at the ventral side of the forearm, a dorsal part, adapted to be arranged at the dorsal side of the forearm, an attachment band, and a compression element in the shape of an inflatable bladder, adapted to provide pressure to the artery puncture site when the compression element is inflated, wherein the ventral part comprises a compression element part where the compression element is arranged. The ventral part is essentially rigid and has a predetermined shape adapted to the anatomy of the forearm, and the dorsal part is at least partly adapted to be supported against the forearm by a supporting part. The ventral part and the dorsal part being interconnected by a flexible part, such that the ventral part, the flexible part and the dorsal part generally form a U-shape being elastic and form stable, and wherein the attachment band is adapted to adjust the ventral and dorsal parts in relation to each other.

The compression arrangement according to the present invention uses an inflatable compression element to achieve hemostasis, making it possible to adjust the compression with great resolution.

According to a first aspect of the present invention, the compression arrangement provides correct positioning of the inflatable compression element, due to the shape of the attachment band and the slightly tilted compression element. The arrangement will stay firmly in place after being applied, without the attachment band having to be overly tightened.

According to a second aspect of the present invention, the shape of the arrangement eliminates the risk of involuntarily compressing the ulnar artery. This is made possible because of the form stable and elastic U-shaped compression arrangement which provides no, or essentially no pressure to skin in the vicinity of the ulnar artery when the attachment band is tightened around the patient's forearm, and the inflatable compression element is filled with air.

The compression arrangement according to the present invention is designed to provide a high level of patient comfort and it will minimise the risk for complications following venous stasis and compression of the ulnar artery.

### SHORT DESCRIPTION OF THE APPENDED DRAWINGS

FIG. 1 shows a schematic cross-sectional view of the wrist anatomy.

FIG. 2 shows the compression arrangement according to the present invention.

FIG. 3 shows the compression arrangement according to the present invention, when being arranged around a patient's forearm.

FIG. 4 shows the compression arrangement according to a preferred embodiment of the present invention.

FIG. 5 shows the compression arrangement provided with an adjusting screw according to a preferred embodiment of the present invention.

FIG. 6 shows the compression arrangement provided with an adhesive tape according to a preferred embodiment of the present invention.

FIG. 7 shows the compression arrangement comprising an adjustably mounted compression element according to a preferred embodiment of the present invention.

FIG. 8 shows the compression element according to a preferred embodiment of the present invention.

FIG. 9 shows an elevated view of the compression arrangement provided with inflating means.

FIG. 10 shows a block diagram schematically illustrating the method for providing pressure to an artery puncture site.

### DETAILED DESCRIPTION OF PREFERRED EMBODIMENTS OF THE INVENTION

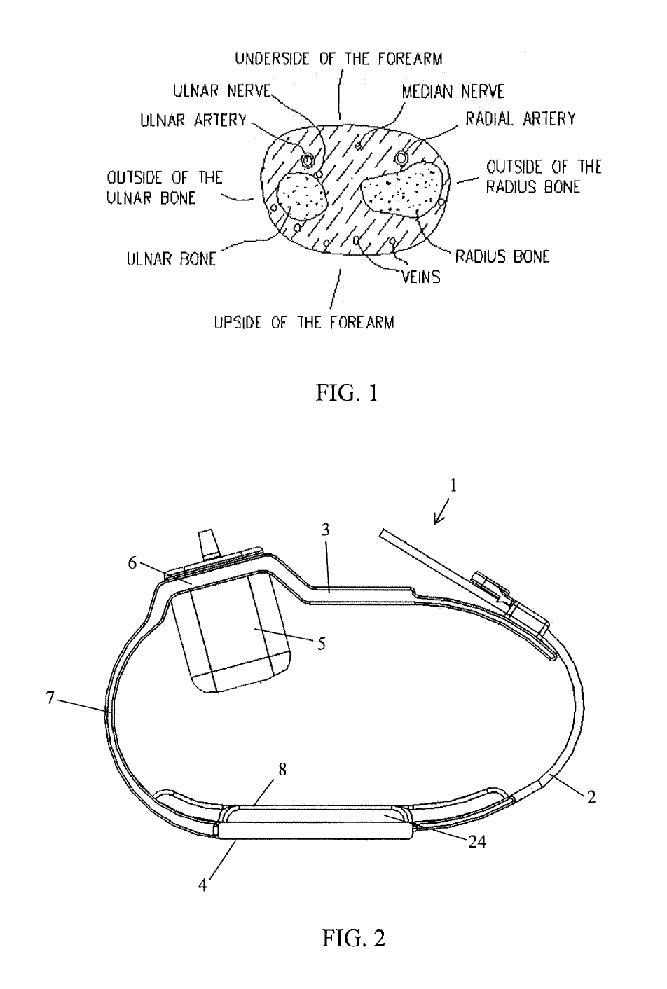

As background information, FIG. 1 shows schematically in cross-section the wrist anatomy with the ulnar and radius bones, the ulnar and radius arteries, the ulnar and median nerves, situated close to the underside of the forearm, i.e. the ventral side of the forearm, and the superficial veins at the upside of the forearm, i.e. the dorsal side of the forearm. In the description below, several references are made to different sides of the forearm, and also these terms are indicated in FIG. 1.

Further, in the description below, the term artery puncture site is used. Herein this term refers to an area surrounding an artery puncture wound.

FIG. 2 illustrates a compression arrangement 1 adapted to be arranged around a patient's forearm (not shown in FIG. 2) to provide pressure to an artery puncture site, according to the present invention. The compression arrangement 1 comprises, a ventral part 3, adapted to be arranged at the ventral side 12 of the forearm 9, a dorsal part 4, adapted to be arranged at the dorsal side 18 of the forearm 9, an attachment band 2, a compression element 5 in the shape of an inflatable bladder, adapted to provide pressure to the artery puncture site 10 when the compression element 5 is inflated, wherein the ventral part 3 comprises a compression element part 6 where the compression element 5 is arranged. The ventral part 3 is essentially rigid and has a predetermined shape adapted to the anatomy of the forearm 9, and the dorsal part 4 is at least partly adapted to be supported against the forearm 9 by a supporting part 8, the ventral part 3 and the dorsal part 4 being interconnected by a flexible part 7, such that the ventral part 3, the flexible part 7 and the dorsal part 4 generally form a U-shape being elastic and form stable, and wherein the attachment band 2 is adapted to adjust the ventral and dorsal parts 3, 4 in relation to each other.

According to one embodiment, the compression arrangement 1 is mainly form stable at the ventral part 3 and dorsal part 4 and elastic mainly at the flexible part 7.

As further is shown in FIG. 2, the flexible part 7 is thinner, less rigid, than the ventral part 3, such that said ventral and dorsal parts 3, 4 may move in relation to each other, this gives the U-shaped compression arrangement 1 its flexibility.

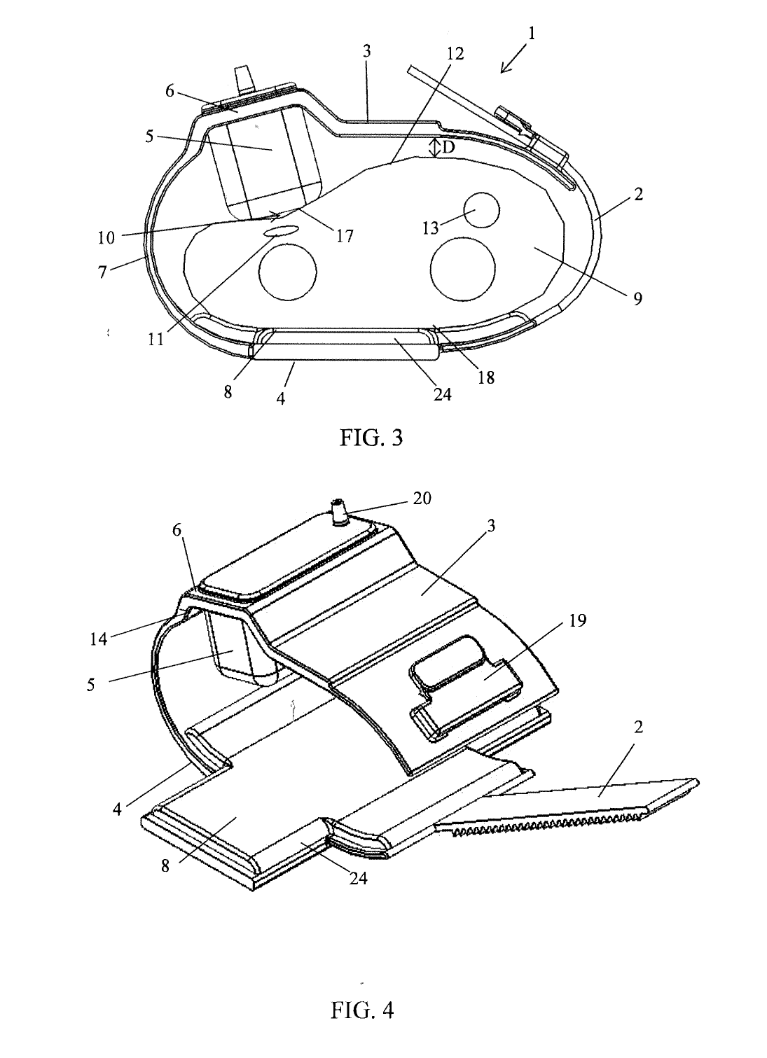

FIG. 3 illustrates the compression arrangement 1 when being arranged around a patient's forearm 9 to provide pressure to a radial artery puncture site 10, and to the radial artery 11. The forearm shown in FIG. 3 is reversed in relation to the forearm shown in FIG. 1.

The ventral part 3, having a predetermined shape adapted to the anatomy of the forearm 9, comprising the compression element part 6 provided with the compression element 5 is arranged at the ventral side 12 of the forearm 9. As seen in FIG. 3, the inflated compression element 5 provides pressure to the radial artery puncture site 10, and the ventral and dorsal parts 3, 4 of the U-shaped elastic and form stable compression arrangement 1 are adjusted in relation to each other by means of the attachment band 2, such that the compression arrangement 1, and in particular the ventral part 3 of the compression arrangement 1, provides no or essentially no pressure to the forearm 9 in the vicinity of the ulnar artery 13. A clearance is thus given to the ulnar artery 13 in the forearm 9, and according to a preferred embodiment of the present invention, there is a distance D between parts of the ventral part 3 of the compression arrangement 1 and the forearm 9 when said attachment band 2 is tightened.

In FIG. 3, the compression arrangement 1 provides pressure to the radial artery 11. However, the compression arrangement 1 may also be arranged to provide pressure to the ulnar artery 13, and then consequently, provides no or essentially no pressure to the radial artery 11.

The compression element 5 is preferably made from a thermoplastic elastomer, such as ethylene vinyl acetate, or silicone, or any other suitable material.

As illustrated in FIG. 4, the compression element part 6 is arc-shaped and at least partly encloses the compression element 5 being arranged at an inward facing surface 14, adapted to face the forearm 9, of the compression element part 6. As also seen in FIG. 4, the supporting part 8 of the compression arrangement 1 is partly wider than the rest of the compression arrangement 1. The wide part distributes the pressure and makes the compression arrangement 1 more comfortable and helps the arrangement 1 to stay firmly in place when being arranged around a patients forearm.

According to another embodiment, the compression arrangement 1 comprises, or may be attached to, a pressure distribution and support plate (not shown), for further distributing the pressure.

In the preferred embodiment of the present invention, shown in FIG. 4, the attachment band 2 and the ventral part 3 are overlapping, and the attachment band 2 and said ventral part 3 are fastened to each other by means of a snap fastener 19. However, the attachment band 2 may, as an obvious constructional variation, be attached to the ventral part 3, and thus be adapted to be overlapping with and fastened to the dorsal part 4. In FIG. 4, it is further shown that the compression element 5 comprises a connector 20.

According to one embodiment of the present invention, the dorsal part 4 is semirigid. However, the dorsal part 4 may, as an obvious constructional variation, be rigid or soft. According to a preferred embodiment illustrated in FIG. 5, the snap fastener 19 is adjustable, preferably by means of an adjusting screw 31. By adjusting the adjusting screw 31, the attachment band 2 is tightened, or loosened, and thus the ventral and dorsal parts 3, 4 are adjusted in relation to each other.

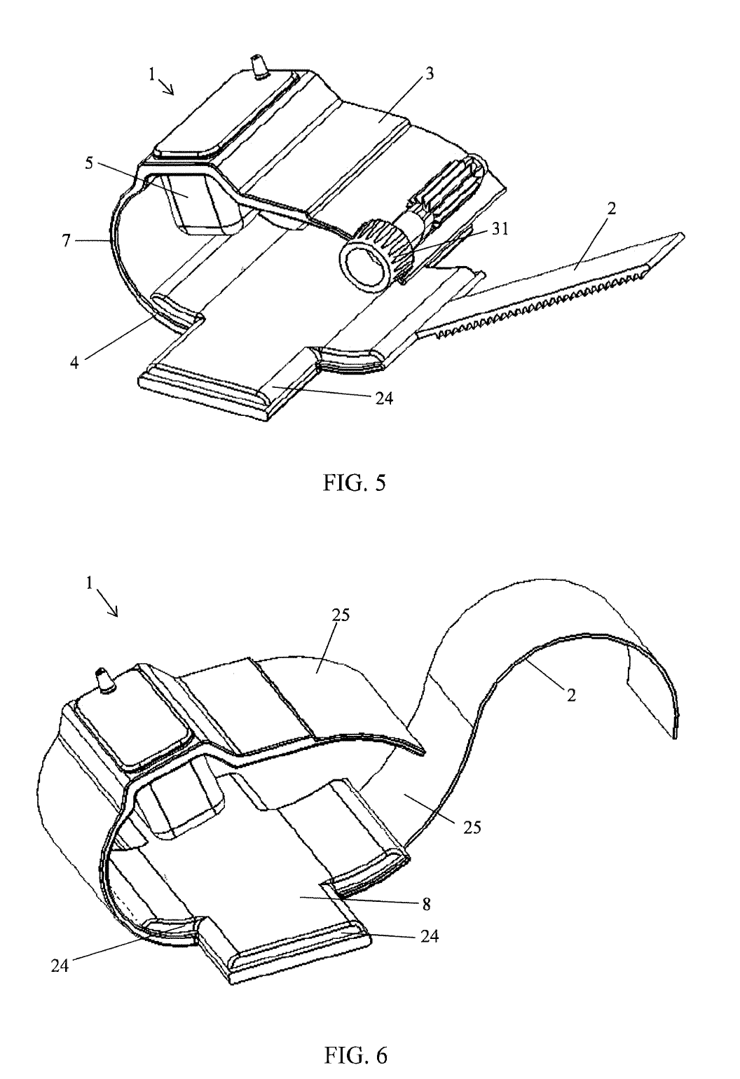

According to another preferred embodiment illustrated in FIG. 6, the attachment band 2 and the ventral part 3 are fastened to each other by means of an adhesive tape 25. The adhesive tape 25 also helps the arrangement 1 to stay firmly in place when being arranged around a patients forearm. Other types of fasteners may also be used, such as Velcro®, or similar.

In one embodiment of the present invention, shown in FIG. 3, a contact surface 17 of the compression element 5 is adapted to be tilted in relation to the ventral side 12 of the forearm 9. Thereby, the compression element 5 is adapted to apply pressure to the radial artery 11 in an inclined direction, such that haemostasis effectively is obtained.

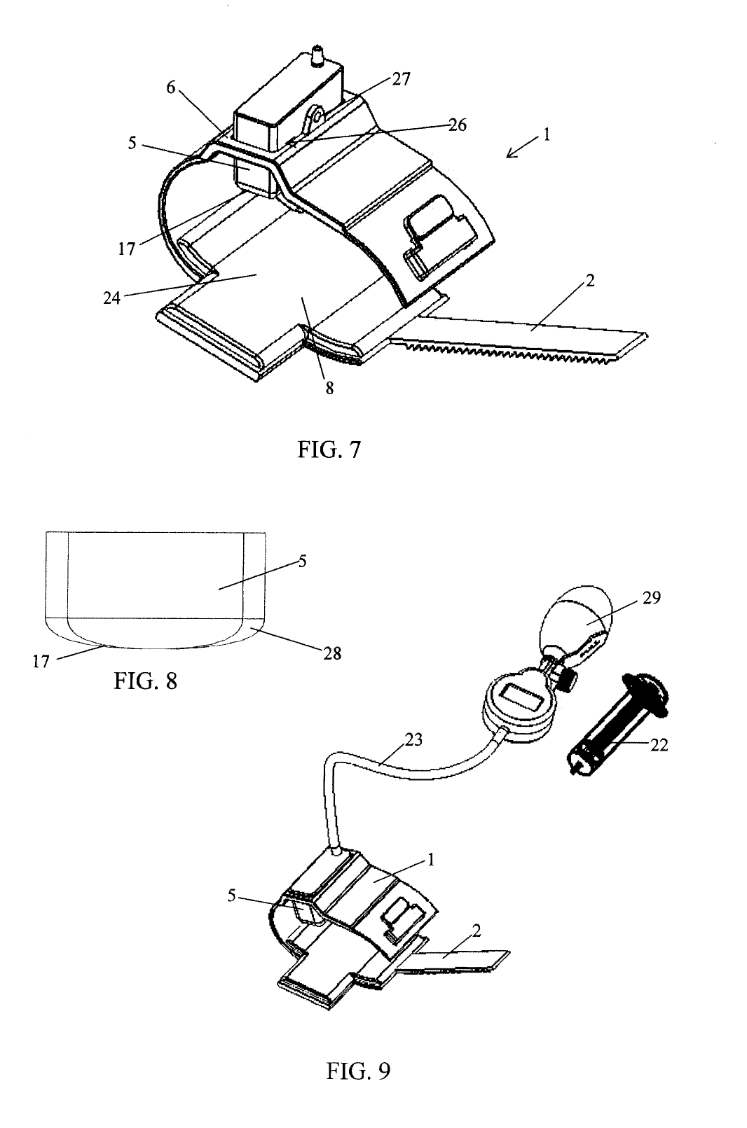

According to the embodiment illustrated in FIG. 7, the compression element 5 is adjustably mounted at the compression element part 6. The compression element part 6 comprises an opening 26 at which said compression element 5 is mounted on a pivot axle 27.

In FIG. 8, the compression element 5 is shown, according to a preferred embodiment of the present invention. The compression element 5 comprises an outer casing 28, and the thickness of the casing 28 varies in an area comprising said contact surface 17. As shown in FIG. 8, the casing 28 is thinner close to the centre of the compression element 5 and thicker further away from the centre.

According to one embodiment shown in FIG. 9, the compression arrangement 1 comprises inflating means 22, 29, for inflating the compression element 5, and the inflating means 22, 29 is connected to the compression element 5 via a tube 23. The inflating means 22, 29 may be a pump 29 or a syringe 22, preferably an injection pressurizer. The pump may be a blood pressure hand pump adapted to inflate the compression element 5. As illustrated in FIG. 9, the compression arrangement 1 may be provided with an indication device, for indicating the present pressure within the compression element 5.

As further shown in FIG. 9, the inflating means 22, 29 is connected to the connector 20 via a tube 23, and the inflatable compression element 5 is filled with air, or any other suitable fluid, by the use of the inflating means 22, 29.

According to one embodiment of the present invention, the compression arrangement 1 is provided with a clamp (not shown) for holding a syringe 22 at the ventral part 3. The clamp holds the syringe 22 in place, while the patient is unobserved, and keeps the syringe 22 from being lost and keeps it permanently attached to the compression arrangement 1. According to one embodiment of the present invention, the ventral part 3 may be partly curved, such that the ventral part 3 is adapted to the anatomy of the forearm 9. The curved shape helps when positioning of the compression arrangement 1 and gives clearance to the ulnar artery.

According to one embodiment of the present invention the compression arrangement 1 is moulded. The compression arrangement 1 may be made from polypropylene, polyethylene, polycarbonate, ABS, polybutylene terephthalate, polyetheretherketone or any other suitable material.

The dorsal part 4 may be provided with a soft inner layer 24, as illustrated in FIGS. 2-7. The inner layer 24 is preferably made from a cellular polymer, such as polyuretan, silicone, ethylene vinyl acetate, or a thermoplastic elastomer.

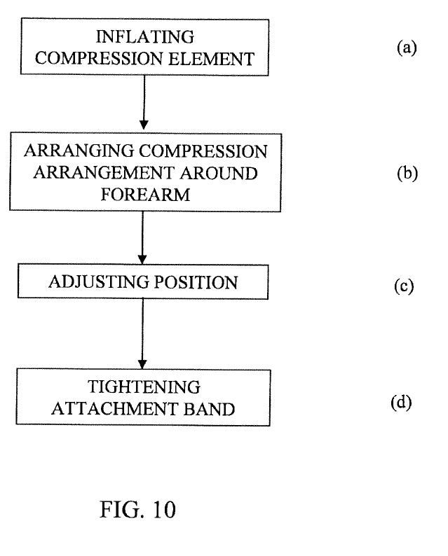

The present invention further relates to a method for providing pressure to an artery puncture site 10 by means of a compression arrangement 1, the method is schematically illustrated in FIG. 10. The method includes;
a) inflating the compression element 5,
b) arranging the compression arrangement 1 around a patient's forearm 9,
c) adjusting the position of the compression arrangement 1 such that the compression element 5 is arranged at the artery puncture site 10,
d) tightening the attachment band 2.

According to one embodiment of the method, steps b), c), and d) is performed prior to step a).

According to another embodiment, the method further includes:
e) adjusting the pressure within the compression element 5.

In one embodiment, the compression element 5 is inflated by means of a syringe 22 without an indication device indicating the pressure within the compression element 5. The method then preferably includes:
a) arranging the compression arrangement 1 around a patient's forearm 9,
b) adjusting the position of the compression arrangement 1 such that the compression element 5 is arranged at the artery puncture site 10,
c) tightening the attachment band 2,
d) inflating the compression element 5,
e) (optionally) adjusting pressure with the adjusting screw 31

Thus, according to this embodiment the compression element 5 is not inflated until the compression arrangement 1 is arranged around the forearm 9.

According to another embodiment, the compression element 5 is inflated by means of a pressurizer, i.e. a syringe provided with an indication device, or a handpump with a manometer. The method then preferably includes:
a) partially inflating compression element 5 (preferably to 20-40 mm Hg higher than the atmospheric pressure)
b) arranging the compression arrangement 1 around a patient's forearm 9,
c) adjusting the position of the compression arrangement 1 such that the compression element 5 is arranged at the artery puncture site 10,
d) tightening the attachment band 2,
e) adjusting pressure in compression element 5 to a specified pressure (typically 20 mm Hg exceeding systolic pressure)

The present invention is not limited to the above-described preferred embodiments. Various alternatives, modifications and equivalents may be used. Therefore, the above embodiments should not be taken as limiting the scope of the invention, which is defined by the appending claims.

1. Compression arrangement (1) adapted to be arranged around a patient's forearm (9) to provide pressure to an artery puncture site (10), said compression arrangement (1) comprises:
a ventral part (3), adapted to be arranged at the ventral side (12) of the forearm (9);
a dorsal part (4), adapted to be arranged at the dorsal side (18) of the forearm (9);
an attachment band (2);
a compression element (5) in the shape of an inflatable bladder, adapted to provide pressure to said artery puncture site (10) when said compression element (5) is inflated;
wherein said ventral part (3) comprises a compression element part (6) where said compression element (5) is arranged,
characterized in that said ventral part (3) is essentially rigid and has a predetermined shape adapted to the anatomy of the forearm (9), said dorsal part (4) at least partly being adapted to be supported against the forearm (9) by a supporting part (8), said ventral part (3) and said dorsal part (4) being interconnected by a flexible part (7), such that said ventral part (3), said flexible part (7) and said dorsal part (4) generally form a U-shape being elastic and form stable, and wherein said attachment band (2) is adapted to adjust said ventral and dorsal parts (3, 4) in relation to each other.

2. Compression arrangement (1) according to claim 1, wherein said flexible part (7) is less rigid than said ventral part (3), such that said ventral and dorsal parts (3, 4) may move in relation to each other.

3. Compression arrangement (1) according to claim 1, wherein said compression element part (6) is arc-shaped and at least partly encloses said compression element (5) being arranged at an inward facing surface (14), adapted to face the forearm (9), of said compression element part (6).

4. Compression arrangement (1) according to claim 1, wherein a contact surface (17) of said compression element (5) is adapted to be tilted in relation to the ventral side (12) of the forearm (9).

5. Compression arrangement (1) according to claim 1, wherein said compression element (5) is adjustably mounted at said compression element part (6).

6. Compression arrangement (1) according to claim 5, wherein said compression element part (6) comprises an opening (26) at which said compression element (5) is mounted on a pivot axle (27).

7. Compression arrangement (1) according to claim 1, wherein said dorsal part (4) is semi-rigid.

8. Compression arrangement (1) according to claim 1, wherein said dorsal part (4) is soft.

9. Compression arrangement (1) according to claim 1, wherein said attachment band (2) and said ventral part (3) are adapted to be overlapping.

10. Compression arrangement (1) according to claim 1, wherein said attachment band (2) and said ventral part (3) are fastened to each other by means of a snap fastener (19).

11. Compression arrangement (1) according to claim 10, wherein said snap fastener (19) is adjustable, preferably by means of an adjusting screw (31).

12. Compression arrangement (1) according to claim 1, wherein said attachment band (2) and said ventral part (3) are fastened to each other by means of an adhesive tape (25).

13. Compression arrangement (1) according to claim 1, wherein said compression arrangement (1) comprises an inflating means (22, 29), for inflating said compression element (5).

14. Compression arrangement (1) according to claim 13, wherein said inflating means (22, 29) is connected to said compression element (5) via a tube (23).

15. Compression arrangement (1) according to claim 1, wherein said dorsal part (4) is provided with a soft inner layer (24).

16. Compression arrangement (1) according to claim 15, wherein said inner layer (24) is made from a cellular polymer.

17. Compression arrangement (1) according to claim 1, wherein said compression arrangement (1) is moulded.

18. Compression arrangement (1) according to claim 1, wherein said compression element (5) is made from a thermoplastic elastomer.

19. Compression arrangement (1) according to claim 4, wherein said compression element (5) comprises a outer casing (28), and wherein the thickness of said casing (28) varies in an area comprising said contact surface (17).

20. Compression arrangement (1) according to claim 1, wherein said compression arrangement (1) comprises a pressure distribution and support plate.

21. Method for providing pressure to an artery puncture site (10) by means of a compression arrangement (1) according to claim 1, the method includes:
a) inflating said compression element (5),
b) arranging said compression arrangement (1) around a patient's forearm (9),
c) adjusting the position of said compression arrangement (1) such that said compression element (5) is arranged at said artery puncture site (10),
d) tightening said attachment band (2).

22. Method according to claim 21, wherein steps b), c), and d) is performed prior to step a).

23. Method according to claim 21, wherein the method further includes:
e) adjusting the pressure within the compression element (5).
